(19) United States
(12) Patent Application Publication
(10) Pub. No.: US 2025/0107860 A1
(43) Pub. Date: Apr. 3, 2025

(54) ENERGY DISCONNECT FOR ROBOTIC SURGICAL ASSEMBLIES

(71) Applicant: Covidien LP, Mansfield, MA (US)

(72) Inventors: Brian Rockrohr, Guilford, CT (US); Jaimeen Kapadia, Cambridge, MA (US); Eric Taylor, West Hartford, CT (US)

(21) Appl. No.: 18/980,695

(22) Filed: Dec. 13, 2024

Related U.S. Application Data
(63) Continuation of application No. 16/643,646, filed on Mar. 2, 2020, now Pat. No. 12,207,894.
(60) Provisional application No. 62/555,936, filed on Sep. 8, 2017.

(51) Int. Cl. A61B 34/37; A61B 34/30; A61B 34/32; A61B 34/35; A61B 46/10

(52) U.S. Cl. CPC A61B 34/37; A61B 34/30; A61B 34/32; A61B 34/35; A61B 46/10

(57) ABSTRACT

A robotic surgical system includes an electrosurgical energy source, an instrument drive unit, a sterile interface module coupled to the instrument drive unit, and a robotic surgical instrument selectively couplable to the sterile interface module. The robotic surgical instrument may be disposed in electrical communication with the electrosurgical energy source while the robotic surgical instrument is coupled to the sterile interface module. The robotic surgical instrument is configured to automatically electrically disconnect from the electrosurgical energy source when the robotic surgical instrument is uncoupled from the sterile interface module.

### TECHNICAL FIELD

The present disclosure relates to robotics, and more specifically to robotic surgical devices, assemblies, and/or systems for performing endoscopic surgical procedures and methods of use thereof.

### BACKGROUND

Robotically-assisted surgery is increasingly being used in minimally invasive medical procedures. Some robotic surgical systems include a console supporting a surgical robotic arm and a robotic surgical instrument mounted to the robotic arm. The robotic surgical instrument may have an elongated shaft that supports at least one end effector (e.g., forceps or a grasping tool) on a distal end thereof.

Although robotically-assisted surgery may have certain advantages over other forms of surgery, robotic surgical systems may reduce tangible feedback that a clinician may otherwise have with a hand-operated surgical instrument. For example, with a hand-operated surgical instrument, a clinician can easily determine (e.g., by visual and/or tactile perception) when an electrosurgical cord is attached and/or powering the hand-operated instrument. With robotic surgical systems, a clinician is often positioned remote from the robotic surgical instrument and may not be able to readily ascertain such tangible feedback, requiring the clinician to be more cognizant of the robotic surgical instrument's electrical connection to an electrosurgical energy source.

In certain instances, this robotic surgical instrument may be removed from the robotic arm during an instrument exchange while still connected to an electrosurgical energy source. The robotic surgical instrument is then placed in the operating theater so that it may be reattached for subsequent reuse. Without having the tangible feedback advantage provided by hand-operated surgical instruments, the clinician is required to take the added step of unplugging the robotic surgical instrument from the electrosurgical energy source in order to avoid inadvertent activation while the robotic surgical instrument is separated from the robotic arm. Also, when reuse is required, besides reconnecting the robotic surgical instrument back to the robotic arm, the clinician is also required to take the additional step of reattaching the robotic surgical instrument to the electrosurgical energy source.

Thus, a need exists for a system that enables a robotic surgical instrument to be efficiently coupled and uncoupled to a robotic arm, and which mitigates the risk of inadvertently activating electrosurgical energy on a robotic surgical instrument.

### SUMMARY

Accordingly, one aspect of the present disclosure is directed to a robotic surgical system. The robotic surgical system includes an electrosurgical energy source, an instrument drive unit, a sterile interface module coupled to the instrument drive unit, and a robotic surgical instrument selectively couplable to the sterile interface module. The robotic surgical instrument may be disposed in electrical communication with the electrosurgical energy source while the robotic surgical instrument is coupled to the sterile interface module. The robotic surgical instrument is configured to automatically electrically disconnect from the electrosurgical energy source when the robotic surgical instrument is uncoupled from the sterile interface module.

In some embodiments, the robotic surgical instrument may include a first electrical connector coupled to the electrosurgical energy source and configured to electrically couple to the sterile interface module.

In certain embodiments, the robotic surgical instrument may include a second electrical connector in electrical communication with an end effector of the robotic surgical instrument. The first and second electrical connectors of the robotic surgical instrument may be electrically isolated from each other when the robotic surgical instrument is uncoupled from the sterile interface module.

In embodiments, an electrical wiring may couple the second electrical connector of the robotic surgical instrument to the end effector.

In some embodiments, the sterile interface module may include first and second electrical connectors configured for electrical communication with the first and second electrical connectors of the robotic surgical instrument.

In certain embodiments, an electrical wiring may couple the first and second electrical connectors of the sterile interface module.

In embodiments, when the robotic surgical instrument is coupled to the sterile interface module, the first and second electrical connectors of the robotic surgical instrument may be in electrical communication with the first and second electrical connectors of the sterile interface module such that the robotic surgical instrument and the sterile interface module form a closed circuit.

In some embodiments, when the robotic surgical instrument is uncoupled from the sterile interface module, the first and second electrical connectors of the robotic surgical instrument may be electrically isolated from the first and second electrical connectors of the sterile interface module.

In certain embodiments, the robotic surgical instrument may include a third electrical connector in electrical communication with the electrosurgical energy source and the first electrical connector of the robotic surgical instrument.

In embodiments, the first electrical connector of the robotic surgical instrument may be a pogo pin.

According to another aspect, the present disclosure is directed to a robotic surgical system, including an electrosurgical energy source, an instrument drive unit, a sterile interface module coupled to the instrument drive unit and including a first electrical connector. The robotic surgical instrument may include a first electrical connector and may be selectively couplable to the sterile interface module. The first electrical connector of the robotic surgical instrument may be configured to couple to the first electrical connector of the sterile interface module when the robotic surgical instrument is coupled to the sterile interface module.

The robotic surgical instrument may be disposed in electrical communication with the electrosurgical energy source while the first electrical connector of the robotic surgical instrument is coupled to the first electrical connector of the sterile interface module. The robotic surgical instrument may be configured to electrically disconnect from the electrosurgical energy source when the robotic surgical instrument is uncoupled from the sterile interface module.

In embodiments, the first electrical connector of the robotic surgical instrument may be coupled to the electrosurgical energy source. The robotic surgical instrument may include a second electrical connector in electrical communication with an end effector of the robotic surgical instrument. The first and second electrical connectors of the robotic surgical instrument electrically may be isolated from each other when the robotic surgical instrument is uncoupled from the sterile interface module.

In some embodiments, an electrical wiring may couple the second electrical connector of the robotic surgical instrument to the end effector.

In certain embodiments, the sterile interface module may include a second electrical connector coupled to the first electrical connector of the sterile interface module.

In embodiments, an electrical wiring may couple the first and second electrical connectors of the sterile interface module.

In some embodiments, a floating plate may be disposed within the sterile interface module. The floating plate may support the first and second electrical connectors and the electrical wiring and may be configured to move from a first position to a second position within the sterile interface module. When the floating plate moves from the first position to the second position, the first and second electrical connectors of the sterile interface module may electrically disconnect from the first and second electrical connectors of the robotic surgical instrument.

In certain embodiments, when the robotic surgical instrument is coupled to the sterile interface module, the first and second electrical connectors of the robotic surgical instrument may be in electrical communication with the first and second electrical connectors of the sterile interface module such that the robotic surgical instrument and the sterile interface module form a closed circuit.

In embodiments, the first and second electrical connectors of the robotic surgical instrument and the sterile interface module may be pogo pins.

According to another aspect of the present disclosure, a method for selectively electrically activating a robotic surgical instrument is provided. The method may include coupling the robotic surgical instrument to an electrosurgical energy source and loading the robotic surgical instrument onto a sterile interface module while the robotic surgical instrument is coupled to the electrosurgical energy source. The method may include electrically coupling a jumper assembly of the sterile interface module to at least one electrical component of the robotic surgical instrument to enable electrosurgical energy to be conducted through the robotic surgical instrument and the sterile interface module upon loading the robotic surgical instrument onto the sterile interface module.

In embodiments, the method may include selectively unloading the robotic surgical instrument from the sterile interface module to automatically electrically deactivate the robotic surgical instrument while the robotic surgical instrument is coupled to the electrosurgical energy source.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description given below, serve to explain the principles of the disclosure, wherein:

FIG. 1 is a schematic illustration of a robotic surgical system in accordance with the present disclosure;

FIG. 2A is a side, elevational view, with parts separated, illustrating an embodiment of a robotic surgical assembly of the robotic surgical system of FIG. 1;

FIG. 2B is a top view of one embodiment of an electromechanical surgical instrument of the robotic surgical assembly shown in FIG. 2A;

FIG. 3 is a perspective view illustrating a portion of the robotic surgical assembly of FIG. 2A with one embodiment of a sterile interface module of the robotic surgical assembly of FIG. 2A coupled to one embodiment of an electromechanical surgical instrument of the robotic surgical assembly of FIG. 2A;

FIG. 4 is a side, elevational view illustrating a sterile interface module coupled to an electromechanical surgical instrument;

FIG. 5A is a side, elevational view illustrating the sterile interface module of FIG. 3 coupled to the electromechanical surgical instrument of FIG. 3;

FIG. 5B is a side, elevational view illustrating the sterile interface module of FIG. 3 separated from the electromechanical surgical instrument of FIG. 3;

FIG. 6A is a bottom view of another embodiment of a sterile interface module; and

FIG. 6B is a side, partial cross-sectional view of the sterile interface module of FIG. 6A as taken along section line 6B-6B shown in FIG. 6A.

### DETAILED DESCRIPTION

Embodiments of the present disclosure are described in detail with reference to the drawings, in which like reference numerals designate identical or corresponding elements in each of the several views. As used herein, the term “distal” refers to that portion of structure that is closer to a patient, while the term “proximal” refers to that portion of structure that is farther from the patient. As used herein, the term “clinician” refers to a doctor, nurse, or other care provider and may include support personnel. In the following description, well-known functions or construction are not described in detail to avoid obscuring the present disclosure in unnecessary detail.

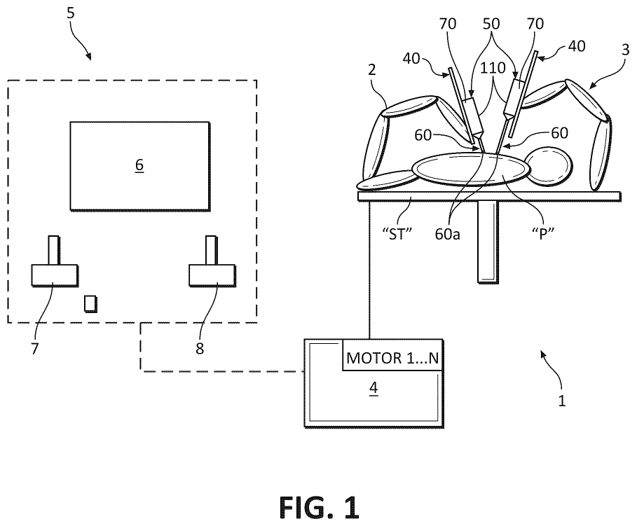

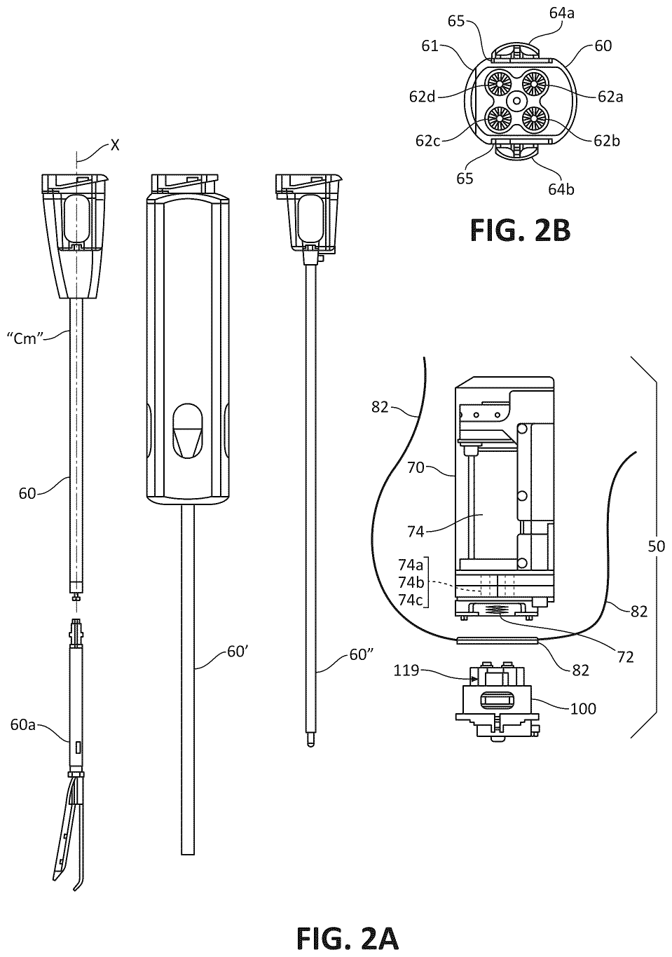

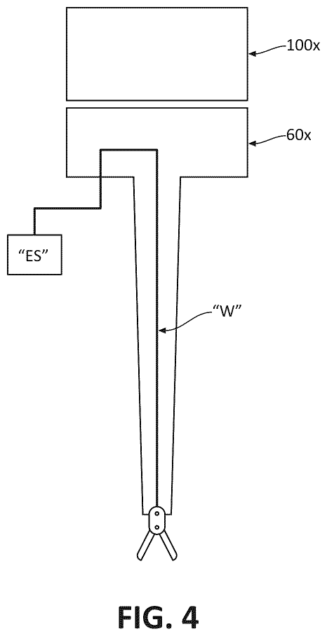

Referring initially to FIGS. 1 and 2A, a surgical system, such as, for example, a robotic surgical system 1, generally includes one or more surgical robotic arms 2, 3, a control device 4, and an operating console 5 coupled with control device 4. Any of surgical robotic arms 2, 3 may have a robotic surgical assembly 50 and an electromechanical surgical instrument 60 coupled thereto. Robotic surgical assembly 50 further includes an instrument drive unit 70 and a collar assembly or sterile interface module, such as sterile interface module 100 or sterile interface module 100x (FIG. 4), that couple to an electromechanical surgical instrument, such as electromechanical surgical instrument 60 or electromechanical surgical instrument 60x (FIG. 4), to instrument drive unit 70.

Surgical system 1 may also include an electrosurgical energy source “ES,” such as a generator, to which the robotic surgical assembly 50, electromechanical surgical instruments 60 (FIG. 2A) or 60x (FIG. 4), instrument drive unit 70, and/or sterile interface modules 100 (FIG. 2A) or 100x (FIG. 4) may be electrically coupled. Although energy source “ES” may include any suitable energy source, for a more detailed description of one example of an electrosurgical generator, reference can be made to U.S. Pat. No. 8,784,410, the entire contents of which are incorporated by reference herein.

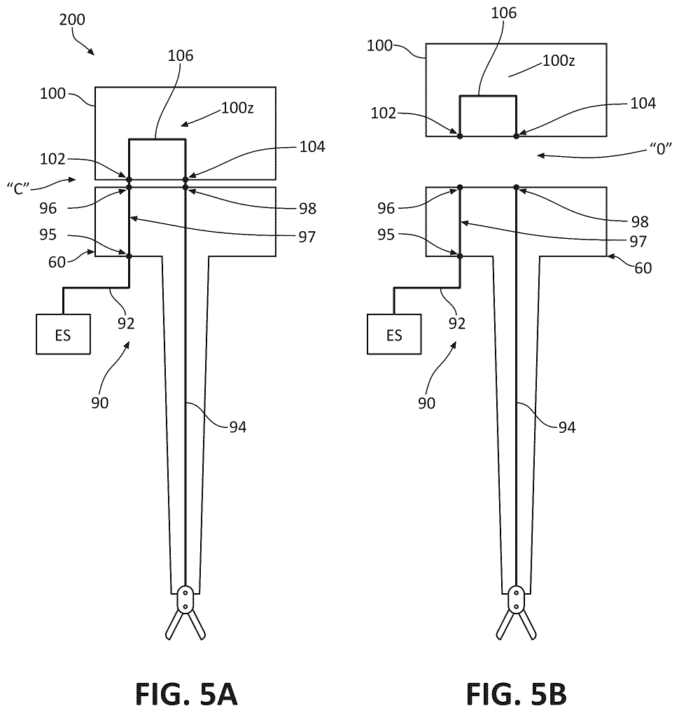

In general, while electromechanical surgical instrument 60x (FIG. 4) may be configured to maintain electrical connection with electrosurgical energy source “ES” when sterile interface module 100x (FIG. 4) and electromechanical surgical instrument 60x are uncoupled, electromechanical surgical instrument 60 (FIG. 2A) may be configured to break electrical connection with electrosurgical energy source “ES” when sterile interface module 100 and electromechanical surgical instrument 60 are uncoupled. More specifically, sterile interface module 100 and electromechanical surgical instrument 60 can be configured to cooperate to provide an electrical disconnect system that electrically disconnects electromechanical surgical instrument 60 from electrosurgical energy source “ES” when sterile interface module 100 and electromechanical surgical instrument 60 are uncoupled (see FIGS. 5A and 5B).

In some embodiments, robotic surgical assembly 50 may be removably attached to a slide rail 40 of one of surgical robotic arms 2, 3. In certain embodiments, robotic surgical assembly 50 may be fixedly attached to slide rail 40 of one of surgical robotic arms 2, 3.

Operating console 5 includes a display device 6, which is configured to display three-dimensional images, and manual input devices 7, 8, by means of which a clinician (not shown), is able to telemanipulate robotic arms 2, 3 in a first operating mode, as known in principle to a person skilled in the art. Each of robotic arms 2, 3 may be composed of any number of members, which may be connected through joints. Robotic arms 2, 3 may be driven by electric drives (not shown) that are connected to control device 4. Control device 4 (e.g., a computer) is set up to activate the drives, for example, by means of a computer program, in such a way that robotic arms 2, 3, attached robotic surgical assembly 50, and thus any attached electromechanical surgical instrument (including an electromechanical end effector thereof configured for activation or firing of an electrosurgical energy-based instrument or the like) execute a desired movement according to a movement defined by means of manual input devices 7, 8. Control device 4 may also be set up in such a way that it regulates the movement of robotic arms 2, 3 and/or of the drives.

Robotic surgical system 1 is configured for use on a patient “P” positioned (e.g., lying) on a surgical table “ST” to be treated in a minimally invasive manner by means of a surgical instrument, e.g., any suitable electromechanical surgical instrument, such as straight/articulatable instruments 60 (e.g., stapling instrument, suturing instrument, electrocautery instrument, etc.), endoscope 60′ or grasper 60″ (FIG. 2A). Robotic surgical system 1 may also include more than two robotic arms 2, 3, the additional robotic arms likewise connected to control device 4 and telemanipulatable by means of operating console 5. A surgical instrument, for example, electromechanical surgical instrument 60, may also be attached to any additional robotic arm(s).

Control device 4 may control one or more motors, e.g., motors (Motor 1 . . . n), each motor configured to drive movement of robotic arms 2, 3 in any number of directions. Further, control device 4 may control instrument drive unit 70 including a motor assembly 74 thereof that drives various operations of an end effector, such as an end effector 60a of electromechanical surgical instrument 60.

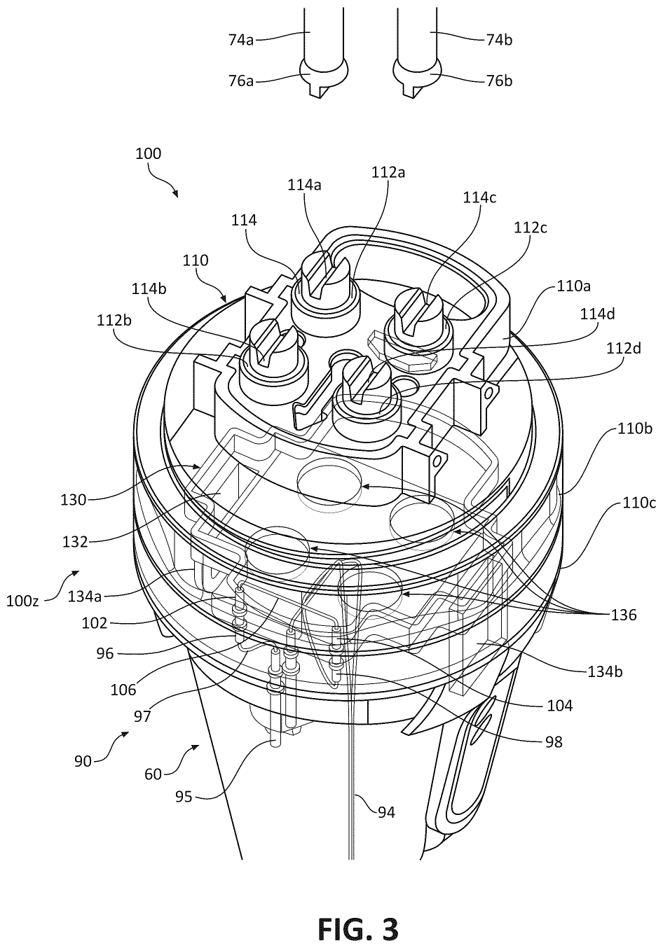

With reference to FIG. 1, motor assembly 74 of robotic surgical assembly 50 includes any number of motors 74a, 74b, 74c, etc. that couple to sterile interface module 100 via a corresponding number of motor couplers 76, such as motor couplers 76a, 76b, 76c, etc. (FIG. 3) extending from motors 74a, 74b, 74c, etc.

In general, robotic surgical assembly 50 transfers power and actuation forces from motors 74a, 74b, 74c, etc. to motor couplers 76a, 76b, 76c, etc. of motor assembly 74, through sterile interface module 100, to driven members 62a, 62b, 62c, etc. (see FIG. 2B) supported within an instrument housing 61 of electromechanical surgical instrument 60. Such transfer of power and actuation forces ultimately drives movement of components of end effector 60a of electromechanical surgical instrument 60 for operating electromechanical surgical instrument 60. This movement may include, for example, a movement of a knife blade (not shown) and/or a closing and opening of jaw members of end effector 60a, an articulation/rotation/pitch/yaw of end effector 60a, and/or the actuation or firing of end effector 60a (e.g. a stapling portion of end effector 60a).

Reference may be made to commonly owned International Patent Application No. PCT/US2014/061329, U.S. Pat. Nos. 8,636,192, or 8,925,786, the entire disclosures of each of which are incorporated by reference herein, for a detailed discussion of illustrative examples of the construction and operation of end effectors for use with, or connection to, the presently disclosed electromechanical surgical instruments.

For a detailed discussion of the construction and operation of a similar robotic surgical system having one or more of the same or similar components for use with one or more components of the presently described robotic surgical system, reference may be made to U.S. Patent Application Publication No. 2012/0116416, the entire disclosure of which is incorporated by reference herein.

With reference to FIG. 2A, instrument drive unit 70 supports sterile interface module 100 for coupling electromechanical surgical instrument 60 to instrument drive unit 70. A distal or leading end portion of instrument drive unit 70 includes one or more buttons 72 that are depressible to selectively attach and/or release sterile interface module 100 to/from instrument drive unit 70.

The distal end portion of instrument drive unit 70 further supports a ring member 80 having a sterile drape 82 secured thereto. Sterile drape 82 is configured to overlie robotic surgical assembly 50 and robotic arms 2, 3 and may be arranged as desired to provide a sterile barrier between the various aforementioned components and/or the surgical site/fluids and electromechanical surgical instrument 60.

With reference to FIGS. 2A and 3, sterile interface module 100 of robotic surgical assembly 50 is provided for selectively interconnecting or interfacing instrument drive unit 70 and an electromechanical surgical instrument such as electromechanical surgical instrument 60. Electromechanical surgical instrument 60 may be laterally coupled (e.g., side-loaded) to, or laterally decoupled from, sterile interface module 100. Advantageously, sterile interface module 100 maintains sterility, provides a means to transmit electrical communication between instrument drive unit 70 and electromechanical surgical instrument 60, provides structure configured to transfer rotational force from instrument drive unit 70 to electromechanical surgical instrument 60 for performing a function with electromechanical surgical instrument 60, and/or provides structure to selectively attach/remove electromechanical surgical instrument 60 to/from robotic surgical assembly 50 (e.g., for rapid instrument exchange).

As seen in FIG. 3, sterile interface module 100 of robotic surgical assembly 50 includes a body member 110 having an upper portion 110a, an intermediate portion 110b, and a lower portion 110c. Body member 110 defines drive transfer channels 112a, 112b, 112c, 112d therethrough that support drive transfer assemblies 114, such as respective drive transfer assemblies 114a, 114b, 114c, 114d, therein. Proximal end portions of drive transfer assemblies 114a, 114b, 114c, 114d of sterile interface module 100 are selectively engagable with respective motor couplers 76a, 76b, 76c, etc. of instrument drive unit 70, and distal end portions of drive transfer assemblies 114a, 114b, 114c, 114d are selectively engagable with respective driven member 62a, 62b, 62c, etc. of an electromechanical surgical instrument, such as electromechanical surgical instrument 60, to selectively operate an end effector 60a of electromechanical surgical instrument 60, for example.

Sterile interface module 100 further includes a floating plate 130 supported between intermediate portion 110b of the body member 110 and lower portion 110c of body member 110. Floating plate 130 includes a base portion 132 and tabs 134a, 134b that extend distally from base portion 132. Tabs 134a, 134b of floating plate 130 extend through lower portion 110c of body member 110. Floating plate 130 defines apertures 136 therein that receive drive transfer assemblies 114a, 114b, 114c, 114d of sterile interface module 100. Floating plate 130 is movable between an uncompressed or extended position and a compressed or retracted position to enable sterile interface module 100 to selectively couple to an electromechanical surgical instrument such as electromechanical surgical instrument 60. Floating plate 130 is spring biased distally toward the uncompressed position by drive transfer assemblies 114a, 114b, 114c, 114d of sterile interface module 100. Moving floating plate 130 from the extended position to the compressed position facilitates a loading and/or unloading of electromechanical surgical instrument 60 onto/from sterile interface module 100 and helps prevent insertion contact/interference between drive transfer assemblies 114 of sterile interface module 100 and corresponding driven members 62a, 62b, 62c, etc. of electromechanical surgical instruments such as electromechanical surgical instrument 60.

With reference to FIGS. 3, 5A, and 5B, body member 110 of sterile interface module 100 supports a jumper assembly 100z having a first electrical connector 102, a second electrical connector 104, and an electrical wiring 106 (e.g., one or more cables, wires, ribbons, jumpers, etc.) that extends between first and second electrical connectors 102, 104 to electrically couple first and second electrical connectors 102, 104 together. Jumper assembly 100z, or components thereof, may be positioned on upper portion 110a, intermediate portion 110b, and/or lower portion 110c of body member 110 of sterile interface module 100.

With reference to FIG. 2B, electromechanical surgical instrument 60 of robotic surgical system 1 generally includes one or more driven members 62a, 62b, 62c, etc. at a first end portion thereof that are coupled to one or more coupling members “CM” (e.g., cables, drive rods, etc.) extending along electromechanical surgical instrument 60 to end effector 60a of electromechanical surgical instrument 60 at a second end portion thereof. Driven members 62a, 62b, 62c, etc. are actuatable to manipulate the one or more coupling members “CM” for operating end effector 60a.

With reference to FIGS. 3, 5A, and 5B, electromechanical surgical instrument 60 includes an instrument electrical assembly 90 having an energy line 92 and an instrument line 94 that are electrically isolated from one another. Energy line 92 is coupled to electrosurgical energy source “ES” and includes a first electrical connector 95, a second electrical connector 96, and a second electrical wiring 97. Energy line 92 couples electrosurgical energy source “ES” to first electrical connector 95 and second electrical wiring 97 of energy line 92 couples first and second electrical connectors 95, 96 together. Instrument line 94 of electrical assembly 90 includes a third electrical connector 98 that electrically couples to one or more components of electromechanical surgical instrument 60, such as end effector 60a of the electromechanical surgical instrument 60.

Electromechanical surgical instrument 60, sterile interface module 100, and electrosurgical energy source “ES” of robotic surgical system 1 collectively define an energy disconnect system 200. Energy disconnect system 200 is configured to enable electromechanical surgical instrument 60 to become electrically active while coupled to electrosurgical energy source “ES” only upon attachment of electromechanical surgical instrument 60 to sterile interface module 100 of robotic surgical assembly 50. In particular, attachment of electromechanical surgical instrument 60 to sterile interface module 100 enables instrument electrical assembly 90 of electromechanical surgical instrument 60 to electrical couple to jumper assembly 100z of sterile interface module 100 so that electrical assembly 90 of electromechanical surgical instrument 60 and jumper assembly 100z of sterile interface module 100 create a continuous circuit in electrical communication with electrosurgical energy source “ES.”

Energy disconnect system 200 is also configured such that if electromechanical surgical instrument 60 of energy disconnect system 200 is disconnected or otherwise removed from sterile interface module 100 of energy disconnect system 200, electromechanical surgical instrument 60 is prevented from receiving electrosurgical energy from electrosurgical energy source “ES” of energy disconnect system 200 so that electromechanical surgical instrument 60 cannot be inadvertently activated. In particular, removal or separation of electromechanical surgical instrument 60 from sterile interface module 100 enables instrument electrical assembly 90 of electromechanical surgical instrument 60 to electrical uncouple or electrically disconnect from jumper assembly 100z of sterile interface module 100 so that the continuous circuit formed by electrical assembly 90 of electromechanical surgical instrument 60 and jumper assembly 100z of sterile interface module 100 becomes electrically discontinuous and/or electrically isolated from electrosurgical energy source “ES.”

In use, with reference to FIGS. 2A and 3, to couple an electromechanical surgical instrument, such as electromechanical surgical instrument 60, to sterile interface module 100, electromechanical surgical instrument 60 is transversely moved (e.g., side loaded) relative to the robotic surgical assembly 50 until electromechanical surgical instrument 60 is fully received or seated in lower portion 110c of sterile interface module 100 whereby energy disconnect system 200 enables the electromechanical surgical instrument 60 to become electrically active.

More specifically, when electromechanical surgical instrument 60 and sterile interface module 60 of robotic surgical assembly 50 are coupled to each other, second electrical connector 96 of electromechanical surgical instrument 60 is releasably connected to first electrical connector 102 of sterile interface module 100. Likewise, second electrical connector 104 of sterile interface module 100 is releasably connected to third electrical connector 98 of electromechanical surgical instrument 60. With respective second and third electrical connectors 96, 98 of electromechanical surgical instrument 60 connected to respective first and second electrical connectors 102, 104 of sterile interface module 100, a closed circuit “C” (FIG. 5A) is formed between electromechanical surgical instrument 60 and sterile interface module 100. Energy from electrosurgical energy source “ES” is routed through the closed circuit “C” to end effector 60a of electromechanical surgical instrument 60.

With robotic surgical assembly 50 of robotic surgical system 1 secured to one of surgical robotic arms 2, 3, of robotic surgical system 1, and electromechanical surgical instrument 60 of robotic surgical system 1 secured to sterile interface module 100 of robotic surgical system 1, a clinician can perform a surgical procedure by robotically controlling driven members 62a, 62b, 62c, etc. of electromechanical surgical instrument 60 with motor assembly 74 of robotic surgical assembly 50 as desired.

To remove electromechanical surgical instrument 60 from robotic surgical assembly 50, for example, to perform an instrument exchange, a clinician can depress paddles 64a, 64b of electromechanical surgical instrument 60 (FIG. 2A). Depression of the paddles 64a, 64b imparts a force on tabs 134a, 134b (FIG. 3) of the floating plate 130 of the sterile interface module 100 to move the floating plate 130 in a proximal direction relative to the body member 110 of sterile interface module 100. As the floating plate 130 moves in a proximal direction, drive transfer shafts 119 of respective drive transfer assemblies 114 translate with floating plate 130 of sterile interface module 100 in the proximal direction against biasing forces from springs (not shown) of respective drive transfer assemblies 114. Movement of drive transfer shafts 119 of respective drive transfer assemblies 114 relative to the body member 110 of sterile interface module 100 separates drive transfer shafts 119 of drive transfer assemblies 114 from respective driven members 62a, 62b, 62c, etc. of electromechanical surgical instrument 60. Once respective drive transfer assemblies 114 are separated from respective driven members 62a, 62b, 62c, etc. of electromechanical surgical instrument 60, electromechanical surgical instrument 60 can be slid laterally out from sterile interface module 100 to remove electromechanical surgical instrument 60 from sterile interface module 100.

When electromechanical surgical instrument 60 is disconnected, decoupled, or otherwise removed from sterile interface module 100 (FIG. 5B), electrical communication between electromechanical surgical instrument 60 and sterile interface module 100 ceases, and energy from the electrosurgical energy source “ES” is no longer provided to end effector 60a of electromechanical surgical instrument 60, even if the electrosurgical energy source “ES” is still powered on. Thus, disconnecting electromechanical surgical instrument 60 from sterile interface module 100 forms a broken or open circuit “O” (FIG. 5B).

Specifically, removing electromechanical surgical instrument 60 from sterile interface module 100 electrically disconnects second electrical connector 96 of electromechanical surgical instrument 60 from first electrical connector 102 of sterile interface module 100 and also disconnects second electrical connector 104 of sterile interface module 100 from third electrical connector 98 of electromechanical surgical instrument 60. Since second electrical connector 96 and third electrical connector 98 of electromechanical surgical instrument 60 are separated or otherwise electrically isolated from each other, electromechanical surgical instrument 60 cannot activate unless connected to sterile interface module 100. More specifically, electromechanical surgical instrument 60 relies on first electrical connector 102 and second electrical connector 104 of sterile interface module 100 to complete the closed circuit “C” (FIG. 5A) and send power from electrosurgical energy source “ES” to end effector 60a of electromechanical surgical instrument 60.

To reestablish the electrical connection (and the closed circuit “C”) between electromechanical surgical instrument 60 and sterile interface module 100, electromechanical surgical instrument 60 can be reattached to sterile interface module 100. Alternatively, a different electromechanical surgical instrument can be attached to the sterile interface module 100.

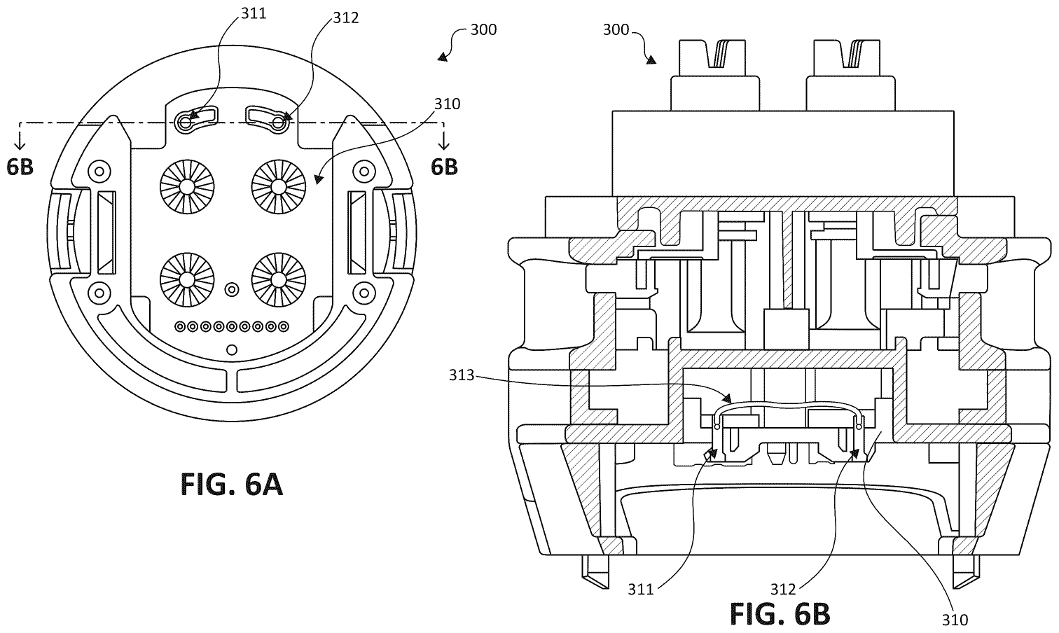

With reference to FIGS. 6A and 6B, provided in accordance with another embodiment of the present disclosure, is a sterile interface module 300. Sterile interface module 300 may be configured for use with robotic surgical assembly 50, an electromechanical surgical instrument such as electromechanical surgical instrument 60, and/or instrument drive unit 70. Sterile interface module 300 may be substantially similar to the sterile interface module 100 described above, except as described herein.

Sterile interface module 300 generally includes a floating plate 310 having a first electrical connector 311 and a second electrical connector 312 that are joined by an electrical wiring 313 to form a jumper assembly 310a. First and second electrical connectors 311, 312 and electrical wiring 313 may be disposed directly on (or in) a surface of floating plate 310. The first and second electrical connectors 311, 312 of floating plate 310 of sterile interface module 300 may be configured to releasably connect, e.g., to respective electrical connectors 96, 98 of electromechanical surgical instrument 60.

In use, as floating plate 310 moves in a proximal direction, electrical connectors 311, 312 of floating plate 310 of sterile interface module 300 are configured to electrically disconnect and/or uncouple from corresponding electrical connectors of an electromechanical surgical instrument, such as electrical connectors 96, 98 of electromechanical surgical instrument 60.

In embodiments, any of the electrical connectors described herein may be electrodes, terminals, contacts, plugs, pogo pins, combinations or variations thereof, or the like. Likewise, any of the electrical wirings described herein may be cables, conductors, wires, jumpers, combinations or variations thereof, or the like. As can be appreciated, any number of electrical connectors, electrical wirings, or combinations or variations thereof, may be used.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. A robotic surgical system, comprising:
an electrosurgical energy source;
an instrument drive unit;
a sterile interface module coupled to the instrument drive unit; and
a robotic surgical instrument selectively couplable to the sterile interface module, the robotic surgical instrument disposed in electrical communication with the electrosurgical energy source while the robotic surgical instrument is coupled to the sterile interface module, the robotic surgical instrument configured to automatically electrically disconnect from the electrosurgical energy source when the robotic surgical instrument is uncoupled from the sterile interface module.
